SEQUENCE LISTING (42 sequences)

SEQ ID NO: 1 - protein, 5 aa, synthetic construct
NAWMH

SEQ ID NO: 2 - protein, 19 aa, synthetic construct
QIKDKSNNYATYYADAVKG

SEQ ID NO: 3 - protein, 13 aa, synthetic construct
VHYGVRFFYTMDV

SEQ ID NO: 4 - protein, 16 aa, synthetic construct
KSSQSLVHSNDNTYLT

SEQ ID NO: 5 - protein, 7 aa, synthetic construct
KVSNRFS

SEQ ID NO: 6 - protein, 9 aa, synthetic construct
GQGTQYPFT

SEQ ID NO: 7 - protein, 5 aa, synthetic construct
SYDVS

SEQ ID NO: 8 - protein, 17 aa, synthetic construct
WVYPVDGSTKFNEKFKG

SEQ ID NO: 9 - protein, 12 aa, synthetic construct
LTYGDFGDAMDY

SEQ ID NO: 10 - protein, 12 aa, synthetic construct
ERKSCVECPPCP

SEQ ID NO: 11 - protein, 124 aa, synthetic construct
EVQLVESGGGLVQPGGSLRLSCAASGFTFSNAWMHWVRQAPGKGLEWVGQIKDKSNNYATYYADAVKGRFTISRDDSKNSLYLQMNSLKTEDTAVYYCRYVHYGVRFFYTMDVWGQGTTVTVSS

SEQ ID NO: 12 - protein, 112 aa, synthetic construct
DIVMTQTPLSLSVTPGQPASISCKSSQSLVHSNDNTYLTWYLQKPGQSPQLLIYKVSNRFSGVPDRFSGSGSGTDFTLKISRVEAEDVGVYYCGQGTQYPFTFGQGTKLEIK

SEQ ID NO: 13 - protein, 121 aa, synthetic construct
QVQLVQSGAEVKKPGASVKVSCKASGYTFTSYDVSWVRQAPGQRLEWMGWVYPVDGSTKFNEKFKGRVTLTADRSSSTAYMELSSLRSEDTAVYYCARLTYGDFGDAMDYWGQGTLVTVSS

SEQ ID NO: 14 - protein, 105 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 15 - protein, 84 aa, Homo sapiens [sequence omitted]

SEQ ID NO: 16 - protein, 96 aa, Macaca fascicularis
QDGNEEMGSITQTPYQVSISGTTVILTCSQHLGSEAQWQHNGKNKEDSGDRLFLPEFSEMEQSGYYVCYPRGSNPEDASHHLYLKARVCENCMEMD

SEQ ID NO: 17 - protein, 84 aa, Macaca fascicularis
FKIPVEELEDRVFVKCNTSVTWVEGTVGTLLTNNTRLDLGKRILDPRGIYRCNGTDIYKDKESAVQVHYRMCQNCVELDPATLA

SEQ ID NO: 18 - protein, 87 aa, Mus musculus
DDAENIEYKVSISGTSVELTCPLDSDENLKWEKNGQELPQKHDKHLVLQDFSEVEDSGYYVCYTPASNKNTYLYLKARVCEYCVEVD

SEQ ID NO: 19 - protein, 84 aa, Mus musculus
FKIQVTEYEDKVFVTCNTSVMHLDGTVEGWFAKNKTLNLGKGVLDPRGIYLCNGTEQLAKVVSSVQVHYRMCQNCVELDSGTMA

SEQ ID NO: 20 - protein, 287 aa, Homo sapiens
TKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWR

SEQ ID NO: 21 - protein, 288 aa, Macaca fascicularis
RTKEDPNAPIRNLRMKEKAQQLMWDLNRNVTDVECIKGTDYSMPAMNNSYCQFGAISLCEVTNYTVRVASPPFSTWILFPENSGTPRAGAENLTCWVHDVDFLSCSWVVGPAAPADVQYDLYLNNPNSHEQYRCLHYKTDARGTQIGCRFDDIAPLSRGSQSSHILVRGRSAAVSIPCTDKFVFFSQIERLTPPNMTGECNETHSFMHWKMKSHFNRKFRYELRIQKRMQPVRTEQVRDTTSFQLPNPGTYTVQIRARETVYEFLSAWSTPQRFECDQEEGASSRAWR

SEQ ID NO: 22 - protein, 315 aa, Mus musculus
SDLAAVREAPPTAVTTPIQNLHIDPAHYTLSWDPAPGADITTGAFCRKGRDIFVWADPGLARCSFQSLSLCHVTNFTVFLGKDRAVAGSIQFPPDDDGDHEAAAQDLRCWVHEGQLSCQWERGPKATGDVHYRMFWRDVRLGPAHNRECPHYHSLDVNTAGPAPHGGHEGCTLDLDTVLGSTPNSPDLVPQVTITVNGSGRAGPVPCMDNTVDLQRAEVLAPPTLTVECNGSEAHARWVARNRFHHGLLGYTLQVNQSSRSEPQEYNVSIPHFWVPNAGAISFRVKSRSEVYPRKLSSWSEAWGLVCPPEVMPVK

SEQ ID NO: 23 - protein, 6 aa, synthetic construct
HHHHHH

SEQ ID NO: 24 - protein, 232 aa, Mus musculus
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 25 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 107 aa, Homo sapiens [sequence omitted]

SEQ ID NO: 33 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 124 aa, synthetic construct
EVQLVETGENLVQPGKSLRLTCATSGFSFSNAWMHWIRQSPEKQLEWVAQIKDKSNNYATYYAESVNGRFTISRDDSKSSIYLHMDNLKEEDSAIYYCRYVHYGVRFFYTMDVWGQGTSVTVSS

SEQ ID NO: 35 - protein, 112 aa, synthetic construct
DVVMTQTPVSLSVSLGDQVSISCRPSQSLVHNNGNTYLSWYLQKPGQSPHPLIYKVSNRFSGISDRFSGSGSGTDFTLKISRVEPDDLGVYYCGQGTQYPFTFGSGTKLEIK

SEQ ID NO: 36 - protein, 248 aa, synthetic construct
QVQLQQSGAELAKPGASVKISCKASGYTFTSYDVSWVKQRPGQGLEWIGWVYPVDGSTKFNEKFKGKATLTADRSSSTAYMQLTSLTSEDSAVYFCARLRYGNYGDAMDDWGQGTSVTVSAGGGGSGGGGSGGGGSDVVMTQTPVSLSVSLGDQVSISCRPSQSLVHNNGNTYLSWYLQKPGQSPHPLIYKVSNRFSGISDRFSGSGSGTDFTLKISRVEPDDLGVYYCGQGTQYPFTFGSGTKLEIK

SEQ ID NO: 37 - protein, 121 aa, synthetic construct
QVQLQQSGAELAKPGASVKISCKASGYTFTSYDVSWVKQRPGQGLEWIGWVYPVDGSTKFNEKFKGKATLTADRSSSTAYMQLTSLTSEDSAVYFCARLRYGNYGDAMDDWGQGTSVTVSA

SEQ ID NO: 38 - protein, 450 aa, synthetic construct
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 39 - protein, 220 aa, synthetic construct
DIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 449 aa, synthetic construct
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 41 - protein, 485 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 220 aa, synthetic construct
DFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0184798 A1
(43) Pub. Date: Jul. 2, 2026

(54) ANTI-CD3 AND ANTI-CD123 BISPECIFIC ANTIBODY AND USE THEREOF

(71) Applicants: Beijing Wisdomab Biotechnology Co., Ltd., Beijing (CN); Genrix (Shanghai) Biopharmaceutical Co., Ltd., Shanghai (CN); Chongqing Genrix Biopharmaceutical Co., Ltd., Chongqing (CN)

(72) Inventors: Zhigang LIU, Beijing (CN); Xiaobo Hao, Beijing (CN); Xueping Zhang, Beijing (CN); Yulan Liu, Beijing (CN); Jingjing Guo, Beijing (CN)

(73) Assignees: Beijing Wisdomab Biotechnology Co., Ltd., Beijing (CN); Genrix (Shanghai) Biopharmaceutical Co., Ltd., Shanghai (CN); Chongqing Genrix Biopharmaceutical Co., Ltd., Chongqing (CN)

(21) Appl. No.: 19/399,869

(22) Filed: Nov. 25, 2025

Related U.S. Application Data
(63) Continuation of application No. 17/797,656, now Pat. No. 12,509,520.

(30) Foreign Application Priority Data
Feb. 5, 2020 (CN) 202010080449.9

(51) Int. Cl. C07K 16/28; A61K 39/00; A61P 35/00; A61P 35/02

(52) U.S. Cl. CPC C07K 16/2866; A61P 35/00; A61P 35/02; C07K 16/2809; A61K 2039/505; C07K 2317/24; C07K 2317/31; C07K 2317/53; C07K 2317/55; C07K 2317/565; C07K 2317/622; C07K 2317/92

(57) ABSTRACT

Provided is a bispecific antibody comprising an antigen-binding portion for human CD3E and/or an antigen-binding portion for human CD123. Further provided are the medical and biological uses of the bispecific antibody.

### CROSS REFERENCE TO RELATED APPLICATION

The present application is a continuation under 35 U.S.C. § 120 of co-pending U.S. application Ser. No. 17/797,656 filed Aug. 4, 2022, which is a U.S. National Stage of International Application No. PCT/CN2020/082151, filed on Mar. 30, 2020, which claims the benefit of and priority to Chinese Patent Application No. 202010080449.9, filed on Feb. 5, 2020, both of which are incorporated herein by reference in their respective entireties.

### SEQUENCE LISTING

The instant application contains a Sequence Listing which has been submitted in XML format via Patent Center and is hereby incorporated by reference in its entirety. Said XML copy, created on Nov. 11, 2025, is named D25C12477US-Sequence-Listing.xml and is 43,762 bytes in size.

### TECHNICAL FIELD

The present application generally relates to the field of antibody drugs. In particular, the present application relates to a bispecific antibody comprising an antigen-binding portion against human CD3E and/or an antigen-binding portion against human CD123 and medical and biological uses thereof.

### BACKGROUND OF THE INVENTION

A bispecific antibody (BsAb) is a type of artificial antibodies that comprise two different antigen binding sites. Bispecific antibodies are widely used in the field of biomedicine, especially tumor immunotherapy. One arm of the bispecific antibodies targeting CD3 can bind to the CD3E subunit in TCR receptor complex on the surface of T cells, and the other arm targets a tumor antigen. In this way, bispecific antibodies can redirect T cells to specifically kill tumor cells in a histocompatibility complex (MHC)-independent manner.

There are numerous platforms for bispecific antibodies and their structures are complex. In terms of antibody structures, the bispecific antibodies can be divided into two categories: those with Fc fragments and those without Fc fragments. The bispecific antibodies without Fc fragments consist of the VH and VL regions or Fab fragments derived from two antibodies. The main representatives of such bispecific antibodies include BiTE, DART™ dual-affinity re-targeting antibody, TandAbs, Bi-Nanobody® (single-domain antibody, VHH), and the like. The advantage of such bispecific antibodies is that there is no mismatch between heavy and light chains. The disadvantages are that their half-life is short and it is inconvenient when they are used in clinical applications. The bispecific antibodies with Fc fragments retain the structures of conventional monoclonal antibodies and can mediate the biological function of the Fc fragment. The representatives of such bispecific antibodies include KIH IgG, crossmab, DVD-Ig™ dual variable domain immunoglobulin, Triomab® trifunctional bispecific antibody, and the like, which have a long half-life in vivo and can have ADCC and CDC activities (see Hongyan Liu, Abhishek Saxena, Sachdev S. Sidhu, et al. Fc engineering for Developing Therapeutic Bispecific Antibodies and Novel Scaffolds. Front. Immunol. 2017; 8: 38).

Therefore, in view of the wide applicability of bispecific antibodies, there is a need to develop new bispecific antibodies in the art.

### SUMMARY OF THE INVENTION

In a first aspect, there is provided in the present application a bispecific antibody comprising an antigen-binding portion against human CD3E, wherein the antigen-binding portion against human CD3E comprises:
HCDR1 (heavy chain CDR1) as set forth in SEQ ID NO:1,
HCDR2 (heavy chain CDR2) as set forth in SEQ ID NO:2,
HCDR3 (heavy chain CDR3) as set forth in SEQ ID NO:3,
LCDR1 (light chain CDR1) as set forth in SEQ ID NO:4,
LCDR2 (light chain CDR2) as set forth in SEQ ID NO:5, and
LCDR3 (light chain CDR3) as set forth in SEQ ID NO:6;
wherein HCDRs and LCDRs are defined according to Kabat.

In a second aspect, there is provided in the present application a bispecific antibody comprising an antigen-binding portion against human CD123, wherein the antigen-binding portion against human CD123 comprises:
HCDR1 (heavy chain CDR1) as set forth in SEQ ID NO:7,
HCDR2 (heavy chain CDR2) as set forth in SEQ ID NO:8,
HCDR3 (heavy chain CDR3) as set forth in SEQ ID NO:9,
LCDR1 (light chain CDR1) as set forth in SEQ ID NO:4,
LCDR2 (light chain CDR2) as set forth in SEQ ID NO:5, and
LCDR3 (light chain CDR3) as set forth in SEQ ID NO:6;
wherein HCDRs and LCDRs are defined according to Kabat.

In a third aspect, there is provided in the present application a bispecific antibody comprising an antigen-binding portion against human CD3E and an antigen-binding portion against human CD123.

In some embodiments of the third aspect, the antigen-binding portion against human CD3E comprises:
HCDR1 as set forth in SEQ ID NO:1,
HCDR2 as set forth in SEQ ID NO:2,
HCDR3 as set forth in SEQ ID NO:3,
LCDR1 as set forth in SEQ ID NO:4,
LCDR2 as set forth in SEQ ID NO:5, and
LCDR3 as set forth in SEQ ID NO:6;
wherein HCDRs and LCDRs are defined according to Kabat.

In some embodiments of the third aspect, the antigen-binding portion against human CD123 comprises:
HCDR1 as set forth in SEQ ID NO:7,
HCDR2 as set forth in SEQ ID NO:8,
HCDR3 as set forth in SEQ ID NO:9,
LCDR1 as set forth in SEQ ID NO:4,
LCDR2 as set forth in SEQ ID NO:5, and
LCDR3 as set forth in SEQ ID NO:6;
wherein HCDRs and LCDRs are defined according to Kabat.

In some embodiments of the third aspect, the antigen-binding portion against human CD3E and the antigen-binding portion against human CD123 comprise the same light chain variable region.

In some embodiments of the third aspect, the bispecific antibody is an IgG1 antibody comprising two heavy chain constant regions having the same hinge region, and the amino acid sequence of the hinge region is set forth in SEQ ID NO:10.

In some embodiments of the third aspect, the bispecific antibody is an IgG1 antibody comprising a first heavy chain constant region and a second heavy chain constant region, wherein the amino acids at positions 354 and 366 of the first heavy chain constant region are C and W, respectively, and the amino acids at positions 349, 366, 368 and 407 of the second heavy chain constant region are C, S, A and V, respectively; wherein the amino acid positions of the antibody constant region are determined according to EU numbering.

In some embodiments of the third aspect, the bispecific antibody is an IgG1 antibody comprising a first heavy chain constant region and a second heavy chain constant region, wherein the amino acids at positions 234, 235 and 331 of the first and second heavy chain constant regions are F, E, and S, respectively; wherein the amino acid positions of the antibody constant region are determined according to EU numbering.

In some embodiments of the first and third aspects, the antigen-binding portion against human CD3E comprises a heavy chain variable region as set forth in SEQ ID NO:11 and a light chain variable region as set forth in SEQ ID NO: 12.

In some embodiments of the second and third aspects, the antigen-binding portion against human CD123 comprises a heavy chain variable region as set forth in SEQ ID NO:13 and a light chain variable region as set forth in SEQ ID NO: 12.

In some embodiments of any one of the above aspects, the antigen-binding portion against human CD3E comprises a single chain fragment variable (scFv) or a Fab fragment.

In some embodiments of any one of the above aspects, the antigen-binding portion against human CD123 comprises a single chain fragment variable (scFv) or a Fab fragment.

In some embodiments of the third aspect, the antibody has a first arm and a second arm, wherein the first arm comprises an antigen-binding portion against human CD3E and the second arm comprises an antigen-binding portion against human CD123:
the first arm comprises the amino acid sequence of the heavy chain variable region as set forth in SEQ ID NO: 11, the amino acid sequence of the heavy chain constant region as set forth in SEQ ID NO:31, the amino acid sequence of the light chain variable region as set forth in SEQ ID NO:12, and the amino acid sequence of the light chain constant region as set forth in SEQ ID NO:32;
the second arm comprises the amino acid sequence of the heavy chain variable region as set forth in SEQ ID NO: 13, the amino acid sequence of the heavy chain constant region as set forth in SEQ ID NO: 30, the amino acid sequence of the light chain variable region as set forth in SEQ ID NO:12, and the amino acid sequence of the light chain constant region as set forth in SEQ ID NO:32.

In a fourth aspect, there is provided in the present application a pharmaceutical composition comprising the bispecific antibody of any one of the first aspect to the third aspect.

In some embodiments of the fourth aspect, the pharmaceutical composition is used for the prevention or treatment of a CD123-positive tumor.

In a fifth aspect, there is provided in the present application use of the bispecific antibody of any one of the first aspect to the third aspect, or the pharmaceutical composition of the fourth aspect in the manufacture of a medicament for the prevention or treatment of a CD123-positive tumor.

In a sixth aspect, there is provided in the present application a method of preventing or treating a CD123-positive tumor comprising administering to a subject in need thereof the bispecific antibody of any one of the first aspect to the third aspect, or the pharmaceutical composition of the fourth aspect.

### BRIEF DESCRIPTION OF THE DRAWINGS

FIG. 1 shows the results of the specific binding of the recombinant anti-CD123 monoclonal antibody to CD123 on the cell surface analyzed by using flow cytometer.

FIG. 2 shows the results of the binding of H7A3 humanized mutant H7A3-h2-m5+L27E5 to CD123 on the surface of MV-4-11 cells analyzed by using flow cytometry.

FIG. 3 shows the results of the binding of the bispecific antibody CD3ExCD123 to the two antigens CD3E and CD123 analyzed by ELISA.

FIG. 4 shows the results of the ability of the bispecific antibody CD3ExCD123 to bind to CD3E on the surface of Jurkat-Dual cells analyzed by using flow cytometer.

FIG. 5 shows the results of the ability of the bispecific antibody CD3ExCD123 to bind to CD123 on the surface of MV-4-11 cells analyzed by using flow cytometer.

FIG. 6 shows the results of the activation of Jurkat-Dual cells by the bispecific antibody CD3×CD123 in the presence of CD123-positive tumor cells, in which Panel A shows the results of the activation of Jurkat-Dual cells by the bispecific antibody CD3×CD123, the anti-CD3E monoclonal antibody H3B8+L27E5, the anti-CD123 monoclonal antibody H7A3-h2-m5+L27E5, and a combination of the anti-CD3E monoclonal antibody H3B8+L27E5 and the anti-CD123 monoclonal antibody H7A3-h2-m5+L27E5; and Panel B shows the results of the activation of Jurkat-Dual cells by the bispecific antibody CD3×CD123, the control bispecific antibody Xmab14045 and HIgG in the presence of CD123-positive target cells, and the results of the activation of Jurkat-Dual cells by the bispecific antibody CD3×CD123 and the control bispecific antibody Xmab14045 in the presence of CD123-negative target cells.

FIG. 7 shows the results of the killing of CD123-positive tumor cells by purified T cells mediated by the bispecific antibody CD3ExCD123.

FIG. 8 shows the results of the specifical activation of T cells and the upregulated expression of CD69 by the bispecific antibody CD3ExCD123 in the presence of CD123-positive tumor cells detected by using flow cytometer.

FIG. 9 shows the results of the proliferation of T cells promoted by the bispecific antibody CD3ExCD123 in the presence of CD123-positive tumor cells detected by using flow cytometer, in which Panel A shows the results of the proliferation of T cells promoted by the bispecific antibody CD3×CD123, the anti-CD3E monoclonal antibody H3B8+L27E5, the anti-CD123 monoclonal antibody H7A3-h2-m5+L27E5, and a combination of the anti-CD3E monoclonal antibody H3B8+L27E5 and the anti-CD123 monoclonal antibody H7A3-h2-m5+L27E5 in the presence of CD123-positive tumor cells; and Panel B shows the results of the proliferation of T cells promoted by the bispecific antibody CD3ExCD123 and the control bispecific antibody Xmab14045 in the presence of CD123-positive tumor cells.

FIG. 10 shows the changes in tumor volume of hCD34+ humanized model mouse with MV-4-11 cells treated with the bispecific antibody CD3ExCD123.

### DESCRIPTION OF SEQUENCES

SEQ ID NO: 1 shows the amino acid sequence of HCDR1 of the heavy chain variable region H3B8 of the anti-human CD3E monoclonal antibody H3B8+L27E5.

SEQ ID NO:2 shows the amino acid sequence of HCDR2 of the heavy chain variable region H3B8 of the anti-human CD3E monoclonal antibody H3B8+L27E5.

SEQ ID NO:3 shows the amino acid sequence of HCDR3 of the heavy chain variable region H3B8 of the anti-human CD3E monoclonal antibody H3B8+L27E5.

SEQ ID NO:4 shows the amino acid sequence of LCDR1 of the light chain variable region L27E5.

SEQ ID NO:5 shows the amino acid sequence of LCDR2 of the light chain variable region L27E5.

SEQ ID NO:6 shows the amino acid sequence of LCDR3 of the light chain variable region L27E5.

SEQ ID NO:7 shows the amino acid sequence of HCDR1 of the heavy chain variable region H7A3-h2-m5 of the anti-human CD123 monoclonal antibody H7A3-h2-m5+L27E5.

SEQ ID NO:8 shows the amino acid sequence of HCDR2 of the heavy chain variable region H7A3-h2-m5 of the anti-human CD123 monoclonal antibody H7A3-h2-m5+L27E5.

SEQ ID NO:9 shows the amino acid sequence of HCDR3 of the heavy chain variable region H7A3-h2-m5 of the anti-human CD123 monoclonal antibody H7A3-h2-m5+L27E5.

SEQ ID NO: 10 shows the amino acid sequence of the hinge region.

SEQ ID NO: 11 shows the amino acid sequence of the humanized heavy chain mutant H3B8 of the rat monoclonal antibody WM03-C6.

SEQ ID NO: 12 shows the amino acid sequence of the humanized light chain mutant L27E5 of the rat monoclonal antibody WM03-C6.

SEQ ID NO: 13 shows the amino acid sequence of the humanized version H7A3-h2-m5.

SEQ ID NO: 14 shows the amino acid sequence of the extracellular region of human (Homo sapiens) CD3E (hCD3E).

SEQ ID NO: 15 shows the amino acid sequence of the extracellular region of human (Homo sapiens) CD3D (hCD3D).

SEQ ID NO: 16 shows the amino acid sequence of the extracellular region of cynomolgus monkey (Macaca fascicularis) CD3E (mfCD3E).

SEQ ID NO: 17 shows the amino acid sequence of the extracellular region of cynomolgus monkey (Macaca fascicularis) CD3D (mfCD3D).

SEQ ID NO:18 shows the amino acid sequence of the extracellular region of mouse (Mus musculus) CD3E (mCD3E).

SEQ ID NO: 19 shows the amino acid sequence of the extracellular region of mouse (Mus musculus) CD3D (mCD3D).

SEQ ID NO:20 shows the amino acid sequence of the extracellular region of human (Homo sapiens) CD123 subtype 1 (hCD123-SP1).

SEQ ID NO:21 shows the amino acid sequence of the extracellular region of cynomolgus monkey (Macaca fascicularis) CD123 subtype 1 (mfCD123-SP1).

SEQ ID NO:22 shows the amino acid sequence of the extracellular region of mouse (Mus musculus) CD123 subtype 1 (mCD123-SP1).

SEQ ID NO:23 shows the amino acid sequence of His tag.

SEQ ID NO:24 shows the amino acid sequence of Fc region of mouse (Mus musculus) antibody IgG2a (mFc).

SEQ ID NO:25 shows the amino acid sequence of the Fc mutant Fck of a heterodimeric human IgG1 subtype.

SEQ ID NO:26 shows the amino acid sequence of the Fc mutant FcH of a heterodimeric human IgG1 subtype.

SEQ ID NO:27 shows the amino acid sequence of the heavy chain constant region of human (Homo sapiens) IgG1 subtype antibody.

SEQ ID NO:28 shows the amino acid sequence of the heavy chain constant region mutant IgG1H of human IgG1 subtype antibody.

SEQ ID NO:29 shows the amino acid sequence of the heavy chain constant region mutant IgG1K of human IgG1 subtype antibody.

SEQ ID NO:30 shows the amino acid sequence of the heavy chain constant region mutant IgG1m3-H of human IgG1 subtype antibody.

SEQ ID NO:31 shows the amino acid sequence of the heavy chain constant region mutant IgG1m3-K of human IgG1 subtype antibody.

SEQ ID NO:32 shows the amino acid sequence of the light chain constant region of human (Homo sapiens) kappa (κ) subtype.

SEQ ID NO:33 shows the amino acid sequence of the light chain constant region of human (Homo sapiens) lambda (λ) subtype.

SEQ ID NO: 34 shows the amino acid sequence of the heavy chain variable region of monoclonal antibody WM03-C6.

SEQ ID NO: 35 shows the amino acid sequence of the light chain variable region of monoclonal antibody WM03-C6.

SEQ ID NO:36 shows the amino acid sequence of the anti-human CD123 single chain fragment variable S8F3.

SEQ ID NO:37 shows the amino acid sequence of the heavy chain variable region S8F3VH of the anti-human CD123 single chain fragment variable S8F3.

SEQ ID NO:38 shows the amino acid sequence of the heavy chain of the anti-CD123 antibody CSL362.

SEQ ID NO:39 shows the amino acid sequence of the light chain of the anti-CD123 antibody CSL362.

SEQ ID NO:40 shows the amino acid sequence of the heavy chain binding to CD123 of Xmab14045 comprising the IgG1m3 subtype with a Hole mutation.

SEQ ID NO:41 shows the amino acid sequence of the scFv structure binding to CD3E of Xmab14045 comprising the IgG1m3 subtype with a Knob mutation.

SEQ ID NO:42 shows the amino acid sequence of the light chain of Xmab14045

### DETAILED DESCRIPTION OF THE INVENTION

### Definitions

The following definitions and methods are provided to better define the present application and guide those of ordinary skill in the art in the practice of the present application. Unless otherwise indicated, the terms used in the present application have the meanings commonly understood by those skilled in the art. All patent documents, academic papers, and other publications cited herein are incorporated by reference in their entirety.

When the structures of antibodies are described herein, reference is made to the EU numbering definition of the human IgG1 antibody with respect to the description on amino acid position numbering, which is well known and readily available to those skilled in the art. Furthermore, where a mutation is described herein in connection with the EU numbering position, it refers to a mutation produced relative to the native antibody sequence.

As used herein, the term “Fc fragment”, “Fc domain”, “Fc portion” or similar terms refer to a portion of the heavy chain constant region of an antibody, including the hinge region (hinge), the CH2 and CH3 segments of the constant region. With reference to the EU numbering definition of the human IgG1 antibody, the Fc fragment refers to the amino acid sequence at positions 216-447 in the constant region of the antibody.

As used herein, the term “Fab (fragment antigen-binding) fragment”, “Fab portion”, or similar terms refer to an antibody fragment capable of binding to an antigen that is produced by treatment of an intact antibody with papain, including the intact light chain (VL-CL), the heavy chain variable region, and the CH1 fragment (VH-CH1).

As used herein, the term “single chain fragment variable (scFv)” refers to an antibody having a single chain structure comprising a polypeptide chain comprising a heavy chain variable region (VH) and a light chain variable region (VL), which is generally constructed using genetic engineering techniques. A flexible linker is typically designed between the heavy chain variable region and the light chain variable region so that the heavy chain variable region and the light chain variable region can be folded into the correct conformation capable of binding to an antigen.

As used herein, the term “antigen-binding portion” refers to a portion of the antibody structure that determines the antigen-binding ability. It will be understood by those skilled in the art that the major parts of an antibody structure that determine the antigen-binding ability are the CDRs, so the CDRs are also the core components of the antigen-binding portion. In the construction of a bispecific antibody, the examples of the “antigen-binding portion” include, but are not limited to a single chain fragment variable (scFv) or a Fab fragment.

As used herein, the term “bispecific antibody” refers to an antibody having the ability to bind to two different antigens, which can consist of two Fc fragments and two antigen-binding portions fused thereto, respectively.

In some embodiments, “a bispecific antibody” used herein refers to a bispecific antibody based on the human IgG1 antibody, and in addition to the altered structures described herein, it has the basic characteristics and function of the human IgG1 antibody. It is well-known to those skilled in the art that “a bispecific antibody” used herein can also be those based on other immunoglobulin subtypes, such as the human IgG2 antibody.

It is well known to those skilled in the art that the complementarity determining regions (CDRs, generally including CDR1, CDR2 and CDR3) are the regions of a variable region that have mostly impact on the affinity and specificity of an antibody. The CDR sequences of a VH or a VL have two common definitions, i.e., the Kabat definition and the Chothia definition (see, e.g., Kabat, “Sequences of Proteins of Immunological Interest”, National Institutes of Health, Bethesda, Md. (1991); A1-Lazikani et al., J. Mol. Biol. 273:927-948 (1997); and Martin et al., Proc. Natl. Acad. Sci. USA, 86:9268-9272 (1989)). For the variable region sequences of a given antibody, the sequences of CDR regions in the VH and the VL can be determined according to the Kabat definition or the Chothia definition. In an embodiment of the present application, CDR sequences are defined according to the Kabat definition.

For the variable region sequences of a given antibody, the sequences of CDR regions in the variable region sequences can be analyzed in a variety of ways, for example, using online software Abysis (http://www.abysis.org/).

As used herein, the term “specific binding” refers to a non-random binding reaction between two molecules, e.g., binding of an antibody to an antigen epitope.

CD3 molecule is an important differentiation antigen on T cell membrane and is also a characteristic marker of the mature T cells. CD3 molecule consists of four chains of γ, δ, ε and ζ, or five chains of γ, δ, ε, ζ and η (ζ and η are homologous isomers), is composed of three dimers of CD3γε, CD3δε and CD3ζζ (or CD3ζη) and expressed on T cell membrane. Three chains of CD3γ, δ and ε contain highly conserved acidic amino acid residues (glutamic acid in γ chain, and aspartic acid in δ and ε chains), which can be non-covalently linked to the basic amino acid residues on the α and β chains of the T cell receptor (TCR) by a salt bridge to form a stable TCR-CD3 complex structure. The complex can transmit T cell activation signals and stabilize the TCR structure. The intracellular domains of each chain of CD3 contain an ITAM (immune receptor tyrosine-based activation motif) structure, which is the basis for a CD3 molecule-mediated intracellular signaling. When the TCR specifically recognizes and binds to an antigen (an antigenic peptide presented by an MHC molecule), tyrosine protein kinases within T cells phosphorylate tyrosine residues on ITAM, and recruit tyrosine protein kinases containing SH2 domains (ZAP-70), thereby transducing the signal into the cytoplasm of T cells to initiate activation mechanism within the cells. Therefore, CD3 has the function of transmitting the activation signal generated after TCR recognizes the antigen and the signal is the first signal for inducing T cell activation.

CD123, also known as human interleukin-3 (IL-3) receptor alpha chain, is a member of the cytokine receptor superfamily with a molecular weight of about 40 KDa and belongs to a type I transmembrane glycoprotein. The interleukin-3 receptor is a heterodimer composed of an alpha chain (CD123) and a beta chain (CD131). After the binding of IL-3 to CD123, CD131 provides signal transduction, which in turn regulates the function of hematopoietic cells and immune cells and stimulates the proliferation of endothelial cells (Testa et al., Biomark Res. 2:4 (2014)).

CD123 is mainly expressed in a myeloid progenitor cell, a plasmacytoid dendritic cell, a monocyte, a basophil and a small subset of B cells (Munoz L et al., Haematologica. 86 (12): 1261-9). About 80% of AML patients have blasts that overexpress CD123, and studies have shown that overexpression of CD123 antigen corresponds to poor prognosis and lower remission rates in AML (Testa U et al., Blood. 2002; 100 (8)). Although most AML patients respond well to initial therapy, some (60-80%) require consolidation therapy to achieve complete remission.

In a first aspect, there is provided in the present application a bispecific antibody comprising an antigen-binding portion against human CD3E, wherein the antigen-binding portion against human CD3E comprises:
HCDR1 as set forth in SEQ ID NO:1,
HCDR2 as set forth in SEQ ID NO:2,
HCDR3 as set forth in SEQ ID NO:3,
LCDR1 as set forth in SEQ ID NO:4,
LCDR2 as set forth in SEQ ID NO:5, and
LCDR3 as set forth in SEQ ID NO:6;
wherein HCDRs and LCDRs are defined according to Kabat.

In a second aspect, there is provided in the present application a bispecific antibody comprising an antigen-binding portion against human CD123, wherein the antigen-binding portion against human CD123 comprises:
HCDR1 as set forth in SEQ ID NO:7,
HCDR2 as set forth in SEQ ID NO:8,
HCDR3 as set forth in SEQ ID NO:9,
LCDR1 as set forth in SEQ ID NO:4,
LCDR2 as set forth in SEQ ID NO:5, and
LCDR3 as set forth in SEQ ID NO:6;
wherein HCDRs and LCDRs are defined according to Kabat.

In a third aspect, there is provided in the present application a bispecific antibody comprising an antigen-binding portion against human CD3E and an antigen-binding portion against human CD123.

In some embodiments of the third aspect, the antigen-binding portion against human CD3E comprises:
HCDR1 as set forth in SEQ ID NO: 1,
HCDR2 as set forth in SEQ ID NO:2,
HCDR3 as set forth in SEQ ID NO:3,
LCDR1 as set forth in SEQ ID NO:4,
LCDR2 as set forth in SEQ ID NO:5, and
LCDR3 as set forth in SEQ ID NO:6;
wherein HCDRs and LCDRs are defined according to Kabat.

In some embodiments of the third aspect, the antigen-binding portion against human CD123 comprises:
HCDR1 as set forth in SEQ ID NO:7,
HCDR2 as set forth in SEQ ID NO:8,
HCDR3 as set forth in SEQ ID NO:9,
LCDR1 as set forth in SEQ ID NO:4,
LCDR2 as set forth in SEQ ID NO:5, and
LCDR3 as set forth in SEQ ID NO:6;
wherein HCDRs and LCDRs are defined according to Kabat.

In some embodiments of the third aspect, the antigen-binding portion against human CD3E and the antigen-binding portion against human CD123 comprise the same light chain variable region.

In some specific embodiments of the third aspect, the antigen-binding portion against human CD3E and the antigen-binding portion against human CD123 comprise the same light chain. This embodiment facilitates proper assembly of the light and heavy chains, and is also a preferred embodiment.

In some embodiments of the third aspect, the bispecific antibody is an IgG1 antibody comprising two heavy chain constant regions having the same hinge region, and the amino acid sequence of the hinge region is set forth in SEQ ID NO: 10, which replaces the sequences at positions 216-230 of the constant region of the natural human IgG1 antibody. The amino acid positions of the antibody constant region are determined according to EU numbering.

In some embodiments of the third aspect, the bispecific antibody is an IgG1 antibody comprising a first heavy chain constant region and a second heavy chain constant region, wherein the amino acids at positions 354 and 366 of the first heavy chain constant region are C and W, respectively, and the amino acids at positions 349, 366, 368 and 407 of the second heavy chain constant region are C, S, A and V, respectively. The amino acid positions of the antibody constant region are determined according to EU numbering.

When a bispecific antibody that retains the Fc domain is constructed, the structure of the bispecific antibody can be optimized from the following two aspects: the first one is the heavy chain heteromerization and the second one is the proper assembly of the light and heavy chains. In some embodiments, two Fc fragments comprise mutations that can ensure heavy chain heteromerization. The KIH (knob-in-hole) technique is a strategy to address heavy chain heteromerization. Generally, the KIH technique refers to the formation of a structure that facilitates pairing of the heterologous halves to each other by modifying the amino acid sequence of the CH3 region, which can maintain the structure of the normal antibody as much as possible while promoting the formation of the bispecific antibody. In some embodiments, the KIH technique utilized includes allowing the amino acids at positions 354 and 366 of one Fc fragment to be C and W, respectively, and the amino acids at positions 349, 366, 368 and 407 of the other Fc fragment to be C, S, A and V, respectively. For guidance on the KIH technique, see, for example, “An efficient route to human bispecific IgG”, A. Margaret Merchant et al., Nature Biotechnology, Volume 16, 1998”, which is incorporated herein by reference in its entirety.

In some embodiments of the third aspect, the bispecific antibody is an IgG1 antibody comprising a first heavy chain constant region and a second heavy chain constant region, wherein the amino acids at positions 234, 235 and 331 of the first and second heavy chain constant regions are F, E, and S, respectively. The amino acid positions of the antibody constant region are determined according to EU numbering.

In some embodiments of the third aspect, the amino acids at positions 234, 235 and 331 of the CH2 fragments of the two heavy chain constant regions are F, E and S, respectively, which can reduce antibody dependent cytotoxicity (ADCC) mediated by the Fc fragment of an antibody, thereby potentially reducing side effects caused by the bispecific antibody in vivo. For guidance on the above mutations, see, for example, “The binding affinity of human IgG for its high affinity Fc receptor is determined by multiple amino acids in the CH2 domain and is modulated by the hinge region”, Stephen M. Canfield et al., J. Exp. Med. Volume 173, 1991, which is incorporated herein by reference in its entirety.

In some embodiments of the first and third aspects, the antigen-binding portion against human CD3E comprises a heavy chain variable region as set forth in SEQ ID NO: 11 (comprising HCDR1 as set forth in SEQ ID NO: 1, HCDR2 as set forth in SEQ ID NO: 2 and HCDR3 as set forth in SEQ ID NO: 3) and a light chain variable region as set forth in SEQ ID NO:12 (comprising LCDR1 as forth in SEQ ID NO:4, LCDR2 as set forth in SEQ ID NO:5 and LCDR3 as set forth in SEQ ID NO:6).

In some embodiments of the second and third aspects, the antigen-binding portion against human CD123 comprises a heavy chain variable region as set forth in SEQ ID NO:13 (comprising HCDR1 as set forth in SEQ ID NO: 7, HCDR2 as set forth in SEQ ID NO: 8 and HCDR3 as set forth in SEQ ID NO:9) and a light chain variable region as set forth in SEQ ID NO:12 (comprising LCDR1 as forth in SEQ ID NO:4, LCDR2 as set forth in SEQ ID NO:5 and LCDR3 as set forth in SEQ ID NO: 6).

In some embodiments of any one of the above aspects, the antigen-binding portion against human CD3E comprises a single chain fragment variable (scFv) or a Fab fragment.

In some embodiments of any one of the above aspects, the antigen-binding portion against human CD123 comprises a single chain fragment variable (scFv) or a Fab fragment.

As a bispecific antibody has two different antigen-binding portions against two different antigens, and the antigen-binding portions can comprise two forms, i.e., a single chain fragment variable (scFv) or a Fab fragment, the configuration of antigen-binding portions of the bispecific antibody can have four combinations for given two antigens: Fab+Fab, Fab+scFv, scFv+Fab, and scFv+scFv.

In some specific embodiments of any one of the above aspects, the antigen-binding portion against human CD3E comprises a Fab fragment and the antigen-binding portion against human CD123 comprises a Fab fragment.

In some specific embodiments of any one of the above aspects, the antigen-binding portion against human CD3E comprises a Fab fragment and the antigen-binding portion against human CD123 comprises a single chain fragment variable (scFv).

In some specific embodiments of any one of the above aspects, the antigen-binding portion against human CD3E comprises a single chain fragment variable (scFv) and the antigen-binding portion against human CD123 comprises a Fab fragment.

In some specific embodiments of any one of the above aspects, the antigen-binding portion against human CD3E comprises a single chain fragment variable (scFv) and the antigen-binding portion against human CD123 comprises a single chain fragment variable (scFv).

The bispecific antibody is also described herein as having two “arms”. The bispecific antibody can be divided into two arms from the central portion. The arms of the bispecific antibody can consist of an Fc fragment and an antigen-binding portion (a Fab fragment or a single chain fragment variable). For the arm consisting of an Fc fragment and a Fab fragment, its structure is similar to that of a common antibody, comprising intact heavy and light chains, and thus the structure of such arm can be represented as Fc+Fab, or can be represented as a heavy chain (Fc+the heavy chain variable region of Fab and CHI fragment)+a light chain (the light chain portion of Fab). When both arms contain the antigen-binding portions in the form of Fab fragment, the structure of the bispecific antibody thus formed is close to that of a native antibody and is a preferred embodiment.

In some embodiments of the third aspect, the antibody has a first arm and a second arm, wherein the first arm comprises an antigen-binding portion against human CD3E and the second arm comprises an antigen-binding portion against human CD123:

the first arm comprises the amino acid sequence of the heavy chain variable region as set forth in SEQ ID NO: 11, the amino acid sequence of the heavy chain constant region as set forth in SEQ ID NO: 31, the amino acid sequence of the light chain variable region as set forth in SEQ ID NO: 12, and the amino acid sequence of the light chain constant region as set forth in SEQ ID NO:32;

the second arm comprises the amino acid sequence of the heavy chain variable region as set forth in SEQ ID NO:13, the amino acid sequence of the heavy chain constant region as set forth in SEQ ID NO: 30, the amino acid sequence of the light chain variable region as set forth in SEQ ID NO: 12, and the amino acid sequence of the light chain constant region as set forth in SEQ ID NO:32.

In some embodiments of any one of the above aspects, the heavy chain constant region of the bispecific antibody is human IgG1 subtype or various mutants of a selected human IgG1 subtype, such as IgG1H, IgG1K, IgG1m3-H, or IgG1m3-K.

In some embodiments of any one of the above aspects, the light chain constant region of the bispecific antibody is human k subtype or human 2 subtype, preferably human k subtype.

In a fourth aspect, there is provided in the present application a pharmaceutical composition comprising the bispecific antibody of any one of the first aspect to the third aspect.

In some embodiments, the pharmaceutical composition further comprises a pharmaceutically acceptable carrier, excipient, diluent, and the like.

In some embodiments, the pharmaceutical composition is used to prevent or treat CD123-positive tumors, for example, acute myeloid leukemia (AML) and blastic plasmacytoid dendritic cell tumors (BPDCN).

In some embodiments, the pharmaceutical composition can further comprise a lubricant, such as talc, magnesium stearate, and mineral oil; a wetting agent; an emulsifier, a suspending agent; a preservative, such as benzoic acid, sorbic acid and calcium propionate; a sweetening agent and/or a flavoring agent and the like.

In some embodiments, the pharmaceutical composition of the present application can be formulated as a tablet, a pill, a powder, a lozenge, an elixir, a suspension, an emulsion, a solution, a syrup, a suppository, or a capsule and the like.

In some embodiments, the pharmaceutical composition of the present application can be delivered using any physiologically acceptable administration route including, but not limited to, oral administration, parenteral administration, nasal administration, rectal administration, intraperitoneal administration, intravascular injection, subcutaneous administration, transdermal administration, inhalation administration and the like.

In some embodiments, a pharmaceutical composition for therapeutic use can be formulated for storage in a lyophilized formulation or in the form of an aqueous solution by mixing an agent with desired purity with a pharmaceutically acceptable carrier or excipient where appropriate.

In a fifth aspect, there is provided in the present application use of the bispecific antibody of any one of the first aspect to the third aspect, or the pharmaceutical composition of the fourth aspect in the manufacture of a medicament for the prevention or treatment of a CD123-positive tumor.

In some embodiments of the fifth aspect, the CD123 positive tumor is selected from the group consisting of acute myeloid leukemia (AML) and blastic plasmacytoid dendritic cell tumors (BPDCN).

In a sixth aspect, there is provided in the present application a method of preventing or treating a CD123 positive tumor, comprising administering to a subject in need thereof the bispecific antibody of any one of the first aspect to the third aspect, or the pharmaceutical composition of the fourth aspect.

In some embodiments of the sixth aspect, the CD123 positive tumor is selected from the group consisting of acute myeloid leukemia (AML) and blastic plasmacytoid dendritic cell tumors (BPDCN).

It is to be understood that the foregoing detailed description is intended only to enable those skilled in the art to have better understanding of the present application and is not intended to limit it in any way. Various modifications and variations can be made to the described embodiments by those skilled in the art.

The following examples are used for purposes of illustration only and are not intended to limit the scope of the present application.

### Examples

### Example 1: Preparation of Recombinant Proteins

Many different recombinant proteins were required in the preparation and identification of a CD3ExCD123 bispecific antibody, including the extracellular region of human CD3E (hCD3E, SEQ ID NO: 14), the extracellular region of human CD3D (hCD3D, SEQ ID NO:15), the extracellular region of monkey CD3E (mfCD3E, SEQ ID NO: 16), the extracellular region of monkey CD3D (mfCD3D, SEQ ID NO: 17), the extracellular region of mouse CD3E (mCD3E, SEQ ID NO: 18), the extracellular region of mouse CD3D (mCD3D, SEQ ID NO:19) and the extracellular region of human CD123 subtype 1 (hCD123-SP1, SEQ ID NO:20), the extracellular region of monkey CD123 subtype 1 (mfCD123-SP1, SEQ ID NO:21), and the extracellular region of mouse CD123 subtype 1 (mCD123-SP1, SEQ ID NO:22). These recombinant proteins all have a large number of post-translational modifications (e.g., glycosylation or disulfide bonds, etc.), and thus the use of the mammal cell expression system would be more advantageous in maintaining the structures and functions of the recombinant proteins. Furthermore, for ease of purification, His tags (SEQ ID NO:23) or Fc fragments of mouse antibody IgG2a (mFc, SEQ ID NO:24) were added to the C-terminus of the non-antibody recombinant proteins, or Fc mutants (FcK, SEQ ID NO:25 or FcH, SEQ ID NO:26) of heterodimeric human IgG1 subtype were formed based on KIH (Knob-Into-Hole) technique. In the preparation of the recombinant antibody, the heavy chain constant region of the antibody can be human IgG1 subtype (SEQ ID NO:27) or various mutants of the selected human IgG1 subtype, such as IgG1H (SEQ ID NO: 28), IgG1K (SEQ ID NO:29), IgG1m3-H (SEQ ID NO:30) or IgG1m3-K (SEQ ID NO:31), and the light chain constant region can be human k subtype (SEQ ID NO:32) or human 2 subtype (SEQ ID NO: 33).

Based on the amino acid sequences of various recombinant proteins of interest recorded in the Uniprot database, the genes (comprising His-tag, mFc or Fc encoding gene) of the above recombinant proteins were designed and synthesized. The synthesized genes encoding various recombinant proteins were cloned into proper eukaryotic expression vectors (such as pcDNA3.1 purchased from Invitrogen Inc.) by conventional molecular biology techniques. Then, liposomes (e.g., 293fectin from Invitrogen Inc.) or other transfection agents (such as PEI) were used to transfect the recombinant protein expression plasmids as prepared into HEK293 cells (such as HEK293F from Invitrogen Inc.). The cells were incubated in suspension under serum-free condition for 3-5 days. Then, the supernatant of the culture was harvested by centrifugation.

For recombinant proteins fused with His-tags, the recombinant proteins in the supernatant were further purified using metal chelate affinity chromatography column (such as HisTrap FF from GE Inc.). The recombinant proteins and antibodies fused with mFc were further purified using a Protein A/G affinity chromatography column (such as Mabselect SURE from GE Inc.). Then, the recombinant protein preservation buffer was then replaced with PBS buffer (pH 7.0) or other suitable buffers using a desalination column (such as Hitrap desaulting from GE Inc.). If necessary, the antibody samples can be sterilized by filtration and then stored in aliquots at −20° C.

### Example 2: Construction of CD123 Mouse Immune Library Based on a Common Light Chain

In order to construct a CD3ExCD123 bispecific antibody based on a common light chain, the light chain variable region of a specific anti-CD3E monoclonal antibody was selected to be matched with the mouse heavy chain variable region that has undergone in vivo affinity maturation of the CD123 antigen. A single chain fragment variable (scFv) library was constructed for screening specific antibodies against CD123 by conventional molecular biology means.

Splenocytes were collected after cross-immunization of 6-8 week old BALB/c mice with hCD123-SP1-His and mfCD123-SP1-His recombinant proteins. Mouse spleen lymphocytes were isolated using mouse lymphocyte separation agent (CAT #DKW33-R0100, DAKEWE). The isolated lymphocytes were subjected to total RNA extraction using total cell RNA extraction kit (CAT #DP430, TIANGEN BIOTECH (BEIJING) CO., LTD.). Using the extracted total RNA as a template, the heavy chain variable region of the antibody was synthesized using a first-strand cDNA synthesis kit (CAT #K1621, Thermo scientific). The light chain variable region of the rat monoclonal antibody WM03-C6 (referring to the sequence of the monoclonal antibody 20E5-F10 in the application WO_2016_116626_A1, in which the amino acid sequence of the heavy chain variable region is set forth in SEQ ID NO: 34, and the amino acid sequence of the light chain variable region is set forth in SEQ ID NO: 35) that specifically recognizes human and cynomolgus monkey CD3E, and the mouse heavy chain variable region, which was obtained by immunizing the mouse with the CD123 recombinant antigen, were obtained by using conventional molecular biology means, such as PCR amplification technique. Then a single chain fragment variable (scFv) was constructed using the overlap extension PCR technique. A scFv library was constructed by cloning the prepared gene encoding the mouse single chain fragment variable into the vector pADSCFV-S(for the experimental technical process, referring to Example 1 of Chinese Patent Application No. 201510097117.0). The capacity of this antibody library reaches 1.2×10E8, and the correct rate is 65%.

### Example 3: Screening of CD123 Mouse Immune Library Having a Common Light Chain

### 3.1 Screening of an Anti-Human CD123 Mouse Single Chain Fragment Variable

The phage library constructed in Example 2 to display mouse single chain fragment variable was screened by the solid-phase screening strategy using the recombinant hCD123-SP1-His prepared in Example 1 as the antigen (for the experimental protocol, referring to Phage Display: General Experimental Guide, Clackson, T. (USA) and Lowman, H. B. (USA) (Ed.); translated by Lan Ma et al., Chemical Industry Press Co., Ltd., May 2008). One single chain fragment variable S8F3 (SEQ ID NO: 36) specifically binding to human CD123 was obtained by carried out a total of three rounds of screening including binding, elution, neutralization, infection and amplification.

The nucleotide sequences encoding the heavy chain variable region S8F3VH of S8F3 (SEQ ID NO: 37) and the light chain variable region WM03-C6VK (SEQ ID NO:35) were cloned into a eukaryotic expression vector (such as pcDNA3.1 from Invitrogen, etc.) fused with the nucleotide sequences encoding a human heavy chain constant region and a light chain constant region using conventional molecular biology means to express the whole antibody S8F3VH+C6VK in combination. Meanwhile, the anti-CD123 antibody CSL362 (the amino acid sequence of the heavy chain was set forth in SEQ ID NO: 38, and the amino acid sequence of the light chain was set forth in SEQ ID NO: 39) was prepared as a positive control antibody for subsequent research with reference to US patent US 2014 0178364 A1.

### 3.2 Affinity Analysis of the Recombinant Anti-CD123 Monoclonal Antibody

The affinity of anti-CD123 antibodies was determined by surface plasmon resonance using Biacore X100. Related reagents and consumables such as Amino Conjugation Kit (BR-1000-50), Human Antibody Capture Kit (BR-1008-39), CM5 Chip (BR100012) and 10×HBS-EP (BR100669) buffer with pH 7.4 were all purchased from GE healthcare. According to the instructions in the kits, 1-Ethyl-3-(3-dimethylaminopropyl) carbodiimide hydrochloride (EDC) and N-Hydroxysuccinimide (NHS) was used to activate the surface of the carboxylated CM5 chip, and the anti-human IgG (Fc) antibody (capture antibody) was diluted to 25 μg/mL with 10 mM, pH5.0 sodium acetate, followed by an injection with a flow rate at 10 μL/min to achieve a coupling volume of approximately 10,000 response units (RU). Following the injection of the capture antibody, 1 M ethanolamine was injected to block unreacted groups. For kinetic measurements, the anti-CD123 antibodies (S8F3VH+C6VK and CSL362) were diluted to 0.5-1 μg/mL and injected at 10 L/min to ensure that about 100RU of the antibody was captured by the anti-human Fc antibody. Then hCD123-SP1-his was diluted to a series of concentration gradients (such as 2.47 nM, 7.4 nM, 22.2 nM, 66.7 nM, and 200 nM), and injected from low to high concentrations at 30 μL/min at 25° C. with a binding time of 120 s and a dissociation time of 600-2400 s. The chip surface was regenerated by injecting 3M MgCl₂ solution at 10 μL/min for 30s. Association rates (Kₒₙ) and dissociation rates (K_{off}) were calculated from association and dissociation sensorgrams fitted by a 1:1 binding model using Biacore X100 evaluation software version 2.0.1. The dissociation equilibrium constant (KD) was calculated as the ratio K_{off}/Kₒₙ. The fitting results are shown in Table 1.

TABLE 1
Affinity constants of the binding of the recombinant
anti-CD123 monoclonal antibody to hCD123-SP1-his

| | Kₒₙ | K_{off} | K_{D} |
|---|---|---|---|
| S8F3VH + C6VK | 4.546E+5 | 3.651E−3 | 8.030E−9 |
| CSL362 | 1.324E+5 | 7.931E−5 | 5.991E−10 |

### 3.3 Identification of the Binding of the Recombinant Anti-CD123 Monoclonal Antibody to Cell Surface CD123 Antigen

KG-1a cells (Human Acute Myeloid Leukemia Cells, purchased from the Cell Resource Center of Institute of Basic Medicine, Chinese Academy of Medical Sciences) at logarithmic phase of growth were used and resuspended to 2×10⁶ cells/mL with PBS buffer containing 1% BSA after centrifugation. KG-1a cells were plated in a 96-well V-bottom plate at 100 μL/well, and the supernatant was removed after centrifugation. The sample S8F3VH+C6VK to be tested, the control sample CSL362 and the irrelevant antibody HIgG (human IgG control (whole molecule), Purifie, A01006, GenScript) were prepared with PBS buffer to final concentrations of 5 μg/mL and 0.5 μg/mL, respectively. The antibodies were added to the wells containing cells and incubated at 4° C. for 1 hr. Then, the plate was washed three times with 200 μL of PBS buffer. The goat anti-human IgG-FITC secondary antibody (ZF-0308, ZSGB-BIO) was added to the wells (100 μL/well), and incubated at 4° C. for 30 minutes in the dark. Then, the cells were washed three times with 200 μL of PBS buffer, and resuspended in 100 μL of PBS buffer to detect FITC channel by flow cytometer (ACEA, Novocyte). The results showed that S8F3VH+C6VK could specifically bind to CD123-positive cell KG-1a (FIG. 1).

### Example 4: Humanization and Activity Identification of WM03-C6 Monoclonal Antibody

### 4.1 Humanized Design of WM03-C6

Humanization studies were performed on the rat monoclonal antibody WM03-C6 to reduce its immunogenicity. The humanization protocol adopted the classic framework transplantation strategy (referring to J immunol. 169, 1119-1125, 2002). The heavy and light chain variable regions of WM03-C6 were respectively compared with the human antibody germline gene sequences in the IMGT database. The appropriate germline gene sequences were selected to provide the framework regions 1 to 3 (FR1+FR2+FR3) of the antibody, and the appropriate J region gene sequence was selected to provide framework region 4 (FR4). This template can be selected based on a variety of factors, such as relative overall length of the antibody, size of the CDRs, amino acid residues located at the junction between the framework regions (FR) and hypervariable regions (CDR) of the antibody, global homology between sequences and the like. The selected template can be a mixture of multiple sequences or can be a consensus template, so long as the proper conformation of the parental complementarity determining regions (CDRs) is maintained as far as possible. At the same time, the mutants of the light and heavy chains of the humanized antibody were designed in order to avoid the possible protein heterogeneity caused by the deamination site NG in the hypervariable regions (CDRs) of the antibody. Finally, a humanized light chain mutant L27E5 (SEQ ID NO: 12) and a humanized heavy chain mutant H3B8 (SEQ ID NO: 11) were obtained.

### 4.2 Affinity Analysis of WM03-C6 Humanized Monoclonal Antibody

Referring to Example 3.2, the anti-human IgG (Fc) antibody was coupled to the surface of CM5 chip, the anti-CD3E antibodies (WM03-C6 and H3B8+L27E5) were diluted to 0.5-1 μg/mL, and injected at 10 μL/min to ensure about 350-400RU of the antibodies were captured by anti-human Fc antibody. Then hCD3E-his was diluted to a series of concentration gradients (such as 6.17 nM, 18.5 nM, 55.6 nM, 167 nM, and 500 nM), and injected from low to high concentration at 30 μL/min at 25° C. with a binding time of 120 s and a dissociation time of 600-1800 s. The chip surface was regenerated by injecting 3M MgCl₂ solution at 10 μL/min for 30s. Association rates (Kₒₙ) and dissociation rates (K_{off}) were calculated from association and dissociation sensorgrams fitted by a 1:1 binding model using Biacore X100 evaluation software version 2.0.1. The dissociation equilibrium constant (K_{D}) was calculated as the ratio K_{off}/Kₒₙ. The fitting results are shown in Table 1.

TABLE 2
Affinity constants of the binding of WM03-C6
humanized monoclonal antibody to hCD3E-his

| | Kₒₙ | K_{off} | KD |
|---|---|---|---|
| WM03-C6 | 2.716E+4 | 2.879E−4 | 1.060E−8 |
| H3B8 + L27E5 | 2.084E+4 | 9.928E−4 | 4.763E−8 |

### Example 5: In Vitro Affinity Maturation of S8F3

The S8F3 heavy chain variable region was subjected to in vitro affinity maturation in order to improve the specificity of the bispecific antibody against the target antigen and enhance the tissue distribution and killing efficiency of the bispecific antibody during administration. A S8F3VH-based CDR3 mutation library was constructed by introducing mutations in the CDR3 of the S8F3 heavy chain variable region using conventional molecular biology means. The designed mutation scheme is shown in Table 3, the library capacity is 1.7×10E8, and the correct rate is 86%.

TABLE 3
The design scheme of S8F3VH-CDR3 mutation library

| Initial amino acid | Mutant amino acid | Degenerate code |
|---|---|---|
| L | L, F, V or I | NTC |
| R | R, T, K, S or N | AVW |
| Y | Y or F | TWC |
| G | G, S, D, N, I or V | RDT |
| N | N, D, T or A | RMC |
| Y | Y or F | TWC |
| G | G, V, A or D | GNT |
| D | D, N, Y, S, T or A | DMC |
| A | A, T, N, D, I or V | RHT |
| M | M, V, T, A, K or E | RHG |
| D | D, N, Y, S, T or A | DMC |
| D | D, Y, F or V | KWT |

Based on the dual-vector system for the phage display (referring to Example 5 in Chinese Patent Application No. 201510097117.0 for the experimental technical process), the constructed S8F3VH-CDR3 mutant library was subjected to three rounds of screening and enrichment with the hCD123-SP1 antigen by solid phase screening method. Finally, the heavy chain variable region mutant H7A3 (SEQ ID NO: 10) with increased affinity was obtained. The obtained nucleotide sequence encoding the H7A3 variable region was cloned into a eukaryotic expression vector fused with the nucleotide sequence encoding the human heavy chain constant region, and combined with the L27E5 light chain expression vector to express the whole antibody.

Referring to Example 3.2, the affinity assay was performed on the S8F3 heavy chain mutant H7A3 using Biacore X100, and the results are shown in Table 4.

TABLE 4
The affinity constants of the binding of the
S8F3 heavy chain mutants to hCD123-SP1-his

| | Kₒₙ | K_{off} | KD |
|---|---|---|---|
| S8F3VH + L27E5 | 4.384E+4 | 1.455E−4 | 3.319E−9 |
| H7A3 + L27E5 | 2.698E+5 | 6.638E−5 | 2.46E−10 |

### Example 6: Humanization of H7A3 and Activity Identification Thereof

A classical framework grafting strategy was used in the humanization of the anti-human CD123 murine monoclonal antibody heavy chain H7A3. Referring to Example 4, CDR transplantation was performed on the heavy chain variable region of H7A3 to obtain a humanized version H7A3VH-h2. At the same time, in order to ensure the conformation and affinity of the antibody, some key amino acids in the framework region of the humanized antibody such as 169, R71, T73, A75 were back mutated, and finally a humanized version H7A3-h2-m5 (SEQ ID NO: 13) was obtained. The antibody variable region gene was designed and synthesized according to the amino acid sequence of the humanized antibody, and cloned into a eukaryotic expression vector to combine with the common light chain L27E5 to express the whole antibody of human IgG1.

Referring to Example 3.2, the affinity assay was performed on H7A3 humanized version H7A3-h2-m5 using Biacore X100, and the results are shown in Table 5.

TABLE 5
The affinity constants of the binding of
H7A3 humanized version to hCD123-SP1-his

| | Kₒₙ | K_{off} | K_{D} |
|---|---|---|---|
| H7A3 + L27E5 | 3.054E+5 | 6.889E−5 | 2.256E−10 |
| H7A3-h2-m5 + L27E5 | 3.144E+5 | 7.671E−5 | 2.440E−10 |

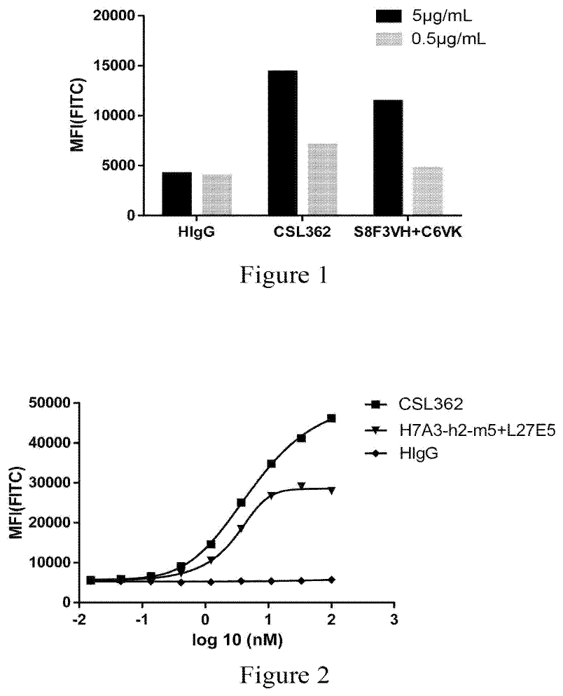

MV-4-11 cells (Human Acute Monocytic Leukemia Cells, purchased from COBIOER BIOSCIENCES CO., LTD) at logarithmic phase of growth were used and resuspended to 2×10⁶ cells/well with PBS buffer containing 1% BSA after centrifugation. MV-4-11 cells were plated in a 96-well V-bottom plate at 100 μL/well, and the supernatant was removed after centrifugation. The sample H7A3-h2-m5+L27E5 to be tested, the control sample CSL362 and the irrelevant antibody HIgG (human IgG control (whole molecule), Purifie, A01006, GenScript) were prepared with PBS buffer to total 9 final concentrations with 100 nM as the starting concentration, followed by a 3-fold gradient dilution. The antibodies were added to the wells containing cells and incubated at 4° C. for 1 hour. Then, the plate was washed three times with 200 μL PBS buffer. The goat anti-human IgG-FITC secondary antibody (ZF-0308, ZSGB-BIO) was added to the wells (100 μL/well), and incubated at 4° C. for 30 minutes in the dark. Then, the cells were washed three times with 200 μL of PBS buffer, and resuspended in 100 μL of PBS buffer to detect FITC channel by flow cytometer (ACEA, Novocyte). The results were shown in FIG. 2, indicating that H7A3-h2-m5+L27E5 can bind to CD123-positive cells MV-4-11 well with a KD value of 3.1 nM.

### Example 7: Preparation of Bispecific Antibodies

The nucleotide sequences encoding the heavy chain variable region H3B8 of the anti-CD3E monoclonal antibody and the heavy chain variable region H7A3-h2-m5 of the anti-CD123 monoclonal antibody were respectively cloned into suitable eukaryotic expression vectors to construct heterodimers based on a common light chain. That is, the nucleotide sequence encoding the heavy chain variable region of the anti-CD3E antibody was cloned into the eukaryotic expression vector fused with the nucleotide sequence encoding the IgG1 constant region with Knob mutation IgG1m3-K, the nucleotide sequence encoding the heavy chain variable region of the anti-CD123 antibody was cloned into the eukaryotic expression vector containing the nucleotide sequence encoding the IgG1 constant region with Hole mutation IgG1m3-H, and the nucleotide sequence encoding the variable region VK of the common light chain L27E5 was cloned into the eukaryotic expression vector fused with the nucleotide sequence encoding the human light chain constant region CK. At the same time, referring to patent WO 2017210443, Xmab14045 based on the same Fc structure (SEQ ID NO: 40, SEQ ID NO: 41, SEQ ID NO: 42) was constructed in order to compare the biological activity of the candidate molecule and the CD3ExCD123 bispecific antibody under clinical research.

The three constructed eukaryotic expression vectors expressing H3B8-IgG1m3-K, H7A3-h2-m5-IgG1m3-H and L27E5-CK were co-transfected into HEK293F cells using liposomes, and the cells were cultured in suspension in a serum-free medium for 3-5 days. The supernatant of the culture was harvested by centrifugation. The bispecific antibodies in the culture supernatant were purified using a Protein A/G affinity chromatography column (e.g., Mabselect SURE, GE Inc.). The recombinant protein preservation buffer was then replaced with PBS buffer (pH 7.0) or other suitable buffers using a desalination column (e.g., Hitrap desaulting, GE Inc.). The desalted protein solution was purified by a size exclusion chromatography (SEC) using Superdex 200 (GE), thereby obtaining the protein of interest. If necessary, the antibody samples can be sterilized by filtration and then stored in aliquots at-20° C. for later use.

### Example 8: Affinity Assay of a Bispecific Antibody

Referring to Examples 3.2 and 4.2, the affinity assays were performed on the anti-CD3E monoclonal antibody H3B8+L27E5, the anti-CD123 monoclonal antibody H7A3-h2-m5+L27E5, the bispecific antibody CD3E×CD123 (CD3×CD123 BsAb) and the control antibody Xmab14045 by surface plasmon resonance technique using Biacore X100. The affinity fitting results are shown in Table 6 and Table 7.

TABLE 6
The affinity constants of the binding of the bispecific
antibody CD3E × CD123 to hCD123-SP1-his

| | Kₒₙ | K_{off} | K_{D} |
|---|---|---|---|
| H7A3-h2-m5 + L27E5 | 2.337E+5 | 5.137E−5 | 2.198E−10 |
| CD3 × CD123 BsAb | 2.254E+5 | 4.015E−5 | 1.781E−10 |
| Xmab14045 | 9.671E+4 | 8.188E−5 | 8.467E−10 |

TABLE 7
The affinity constants of the binding of the bispecific antibody CD3E × CD123 to CD3E

| | hCD3E-his | | | mfCD3E-his | | |
|---|---|---|---|---|---|---|
| | Kₒₙ | K_{off} | K_{D} | Kₒₙ | K_{off} | K_{D} |
| H3B8 + L27E5 | 5.607E+4 | 7.108E−4 | 1.268E−8 | 1.233E+5 | 6.492E−4 | 5.263E−9 |
| CD3 × CD123BsAb | 5.646E+4 | 7.967E−4 | 1.411E−8 | 1.368E+5 | 1.571E−3 | 1.149E−8 |
| Xmab14045 | 5.648E+5 | 3.674E−4 | 6.505E−10 | 8.633E+5 | 8.074E−4 | 9.352E−10 |

### Example 9: Identification of the Ability of the Bispecific Antibody to Simultaneously Recognize Both CD3E and CD123 Antigens

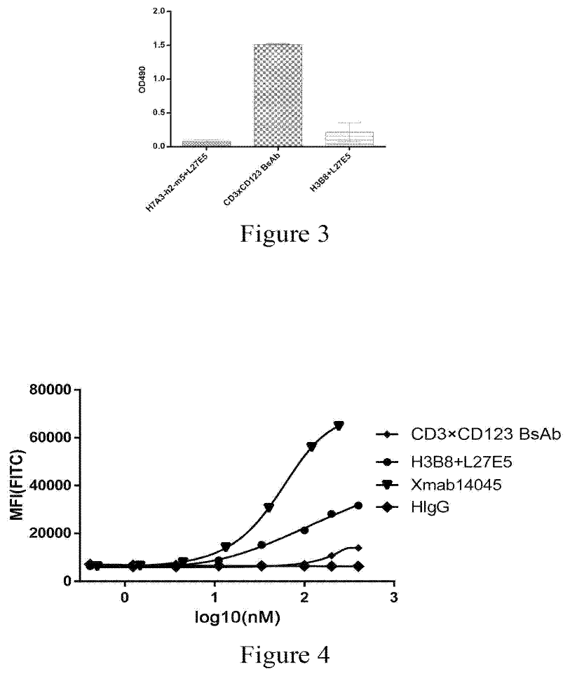

The ability of the bispecific antibody CD3E×CD123 (CD3E×CD123 BsAb) to simultaneously bind to both CD3E and CD123 antigens was detected using conventional ELISA methods. A 96-well ELISA plate was coated with the antigen CD123-SP1-mFc (3 μg/mL, 100 μL/well) overnight at 4° C. After being blocked with blocking solution PBS-0.1% Tween 20-3% milk at 37° C. for 1 hour, the anti-CD123 monoclonal antibody H7A3-h2-m5+L27E5, the anti-CD3E monoclonal antibody H3B8+L27E5 and the bispecific antibody CD3E×CD123 were respectively added to the wells in duplicate (10 μg/mL, 100 μL/well), and incubated at 37° C. for 1 hour. The ELISA plate was washed with PBS-0.1% Tween 20, followed by addition of CD123-His antigen (1 μg/mL, 100 μL/well) and incubation at 37° C. for 1 hour. The ELISA plate was washed with PBS-0.1% Tween 20, followed by addition of HRP mouse anti-his IgG (cw0285M, Beijing Com Win Biotech Co., Ltd.) and incubation at 37° C. for 1 hour. The ELISA plate was washed with PBS-0.1% Tween 20, and OPD substrate color development solution was added. The color development was terminated with 1 M H₂SO₄ after 5-10 minutes. The optical density values at 492 nm/630 nm dual wavelength were measured using a microplate reader. The result of ELISA assay was shown in FIG. 3. The bispecific antibody CD3E×CD123 simultaneously recognized both CD3E and CD123 antigens.

### Example 10: Identification of the Ability of the Bispecific Antibody to Recognize CD3E and CD123 on Cell Surface

### 10.1 Identification of the Ability of the Bispecific Antibody to Recognize CD3E on the Cell Surface

Jurkat-Dual cells (jktd-isnf, purchased from Invivogen) at the logarithmic phase of growth were used and resuspended to 2×10⁶ cells/well with PBS buffer containing 1% BSA after centrifugation. Jurkat-Dual cells were plated in a 96-well V-bottom plate at 100 μL/well, and the supernatant was removed after centrifugation. The control sample of the bispecific antibody Xmab14045, the anti-CD3E monoclonal antibody H3B8+L27E5, the irrelevant antibody HIgG (human IgG control (whole molecule), Purifie, A01006, GenScript) and the bispecific antibody CD3×CD123 (CD3×CD123 BsAb) were diluted with PBS buffer to total 8 concentrations with 400 nM as the starting concentration, followed by 2-fold dilution for the first 3 concentration points and 3-fold dilution for the last 5 concentration points. The antibodies were added to the wells containing cells and incubated at 4° C. for 1 hour. Then, the plate was washed three times with 200 μL PBS buffer. The goat anti-human IgG-FITC secondary antibody (ZF-0308, ZSGB-BIO) was added to the wells (100 μL/well), and incubated at 4° C. for 30 minutes in the dark. Then, the cells were washed three times with 200 μL of PBS buffer, and resuspended in 100 μL of PBS buffer to detect FITC channel by flow cytometer (ACEA, Novocyte). The results showed that the bispecific antibody CD3×CD123 could bind to CD3E on the surface of Jurkat-Dual cells, and the binding strength was weaker than that of the control sample Xmab14045, which is consistent with the affinity measurement results in Example 9 (FIG. 4).

### 10.2 Identification of the Ability of the Bispecific Antibody to Recognize CD123 on the Cell Surface

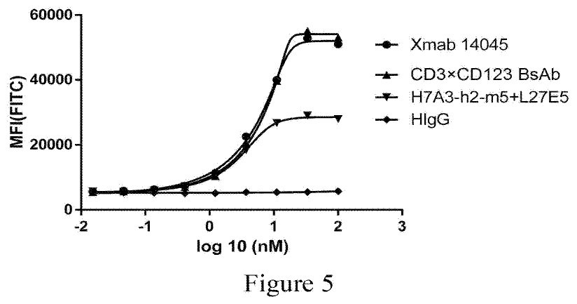

MV-4-11 cells at the logarithmic phase of growth were used and resuspended to 2×10⁶ cells/well with PBS buffer containing 1% BSA after centrifugation. MV-4-11 cells were plated in a 96-well V-bottom plate at 100 μL/well, and the supernatant was removed after centrifugation. The control sample of the bispecific antibody Xmab14045, the anti-CD123 monoclonal antibody H7A3-h2-m5+L27E5, the bispecific antibody CD3×CD123 (CD3×CD123 BsAb) and the irrelevant antibody HIgG (human IgG control (whole molecule), Purifie, A01006, GenScript) were diluted with PBS buffer to total 9 concentrations with 100 nM as the starting concentration, followed by a 3-fold gradient dilution. The antibodies were added to the wells containing cells and incubated at 4° C. for 1 hour. Then, the plate was washed three times with 200 μL PBS buffer. The goat anti-human IgG-FITC secondary antibody (ZF-0308, ZSGB-BIO) was added to the wells (100 μL/well), and incubated at 4° C. for 30 minutes in the dark. Then, the cells were washed three times with 200 μL of PBS buffer, and resuspended in 100 μL of PBS buffer to detect FITC channel by flow cytometer (ACEA, Novocyte). The results showed that the bispecific antibody CD3×CD123 could well bind to CD123 on the surface of CD123-positive MV-4-11 cells with a K_{D} value of 7.08 nM, which was comparable to that of Xmab14045 (K_{D}=6.07 nM) (FIG. 5)

### Example 11: Specific Activation of Jurkat-Dual Cells Mediated by the Bispecific Antibody

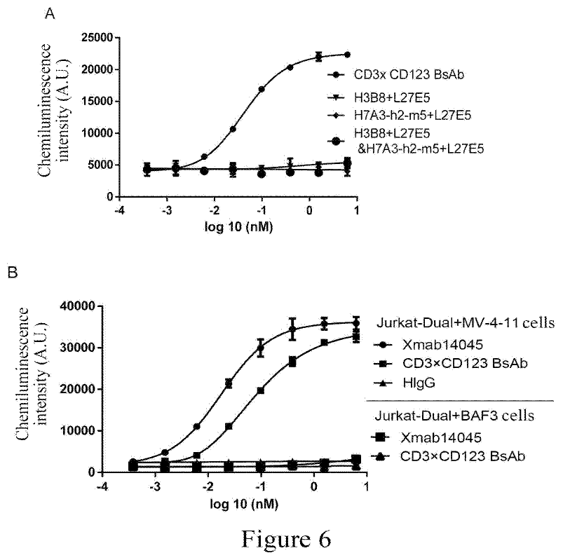

MV-4-11 cells (CD123+ cells) at logarithmic phase of growth were collected. After centrifugation, the cells were resuspended with 1640 medium to 2×10⁶ cells/mL and plated in the cell plate at 50 μL/well. Jurkat-dual cells (purchased from Invivogen) at logarithmic phase of growth were collected, centrifugated, and resuspended with 1640 medium to 2×10⁶ cells/mL, and were added to the cell plate with 50 μL/well to obtain a final E: T ratio of 1:1. Then, total 8 concentrations of the bispecific antibody CD3×CD123 (CD3×CD123 BsAb, 50 μL/well) was added with 6 nM as the starting concentration, followed by a 4-fold gradient dilution. The control sample of the bispecific antibody Xmab14045, the anti-CD3E monoclonal antibody H3B8+L27E5, the anti-CD123 monoclonal antibody H7A3-h2-m5+L27E5, the combination of H3B8+L27E5 and H7A3-h2-m5+L27E5 (H3B8+L27E5&H7A3-h2-m5+L27E5), the CD123 negative target cells (BAF3 cells) and the irrelevant antibody HIgG (human IgG control (whole molecule), Purifie, A01006, GenScript) were set as controls, and the concentration of these controls was the same as that of the bispecific antibody CD3×CD123. After 20 hours of incubation, the supernatant was taken, and the specific activation of Jurkat-Dual cells mediated by tumor cells under different conditions was detected and analyzed according to the QUANTI-Luc™ instructions (QUANTI-Luc, Invivogen, rep-qlc2) (FIGS. 6A-B).

### Example 12: The Expression of the Activation Molecules on the Surface of the Purified T Cells and the Killing of the CD123 Positive Tumor Cells Mediated by the Bispecific Antibody CD3×CD123

### 12.1 Isolation of Human Peripheral Blood Mononuclear Cells (PBMCs)

Blood (50 mL each) was collected from normal volunteers. All of the volunteers had signed informed consent. Inclusion criteria for volunteers were as follow:
1. Age older than 18 years;
2. No HIV and HBV infection;
3. Normal blood routine test;
4. Non-pregnant or non-lactating women.

PBMCs were isolated from whole blood of the volunteers using Ficoll density gradient centrifugation and were cultured in 1640 medium.

### 12.2 Detection of the Killing of the Purified T Cells to the CD123 Positive Tumor Cells Mediated by Bispecific Antibody

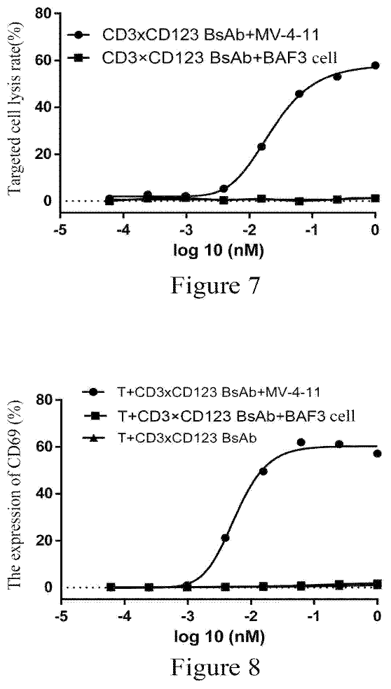

MV-4-11 cells (CD123⁺ cells) at the logarithmic phase of growth were collected, centrifuged, and resuspended in 1640 medium to 1×10⁶ cells/mL, and were plated in cell plates at 50 μL/well. Then, total 8 concentrations of the bispecific antibody CD3×CD123 (CD3×CD123 BsAb, 50 μL/well) was added with 1 nM as the starting concentration, followed by a 4-fold gradient dilution. The purified T cells (5×10⁶ cells/mL, 50 μL/well) were obtained by sorting from PBMCs according to the instructions of the T cell negative sorting kit (BD IMaq human T lymphocyte enrichment set-DM, 557874, BD), and the final effector to target ratio was 5:1. At the same time, a control with only target cells (MV-4-11 cells), a control with only effector cells (T), and a blank control with only medium were set, and the volume was filled to 150 μL with the medium. After 20 hours of incubation, the supernatant was taken. The killing rate of T cells to tumor cells mediated by the bispecific antibody CD3E×CD123 was detected and analyzed with reference to the instructions of the cytoTox96® Non-Radioactive Cytotoxicity Assay (G1780, Promega). The results showed that the bispecific antibody CD3×CD123 could specifically mediate the killing of CD123-positive tumor cells by T cells, but had no killing effect on CD123-negative tumor cells (BAF3 cells) (FIG. 7).

12.3 Identification of the Expression of the Surface Activation Molecule after T Cells being Activated by the Bispecific Antibody

In the section 12.2, the supernatant was removed and the cells were washed twice with PBS buffer, and incubated with the anti-human CD3-APC antibody (ebioscience, 17-0037-42) and the anti-human CD69-PE antibody (ebioscience, 11-0069-42) at 4° C. for 30 minutes in the dark. Then, the cells were washed twice with PBS buffer, resuspended in 100 μL PBS buffer, and loaded to the flow cytometer (ACEA, Novocyte) for detection to compare the expression difference of the activation marker CD69 in the CD3 positive cell population (MV-4-11 cells) before and after treated with the bispecific antibody CD3×CD123 (CD3×CD123 BsAb). The results showed that the bispecific antibody CD3×CD123 specifically up-regulated the expression of CD69 on the surface of T cells in the presence of positive tumor cells, while failing to up-regulate the expression of CD69 on the surface of T cells in the presence of CD123-negative tumor cells (BAF3 cells) (FIG. 8).

### Example 13: Bispecific Antibody-Mediated Proliferation of T Cells In Vitro

### 13.1 Isolation of Human Peripheral Blood Mononuclear Cells (PBMCs)

PBMCs were obtained by sorting according to Example 12.1.

### 13.2 Bispecific Antibody-Mediated Proliferation of T Cells In Vitro

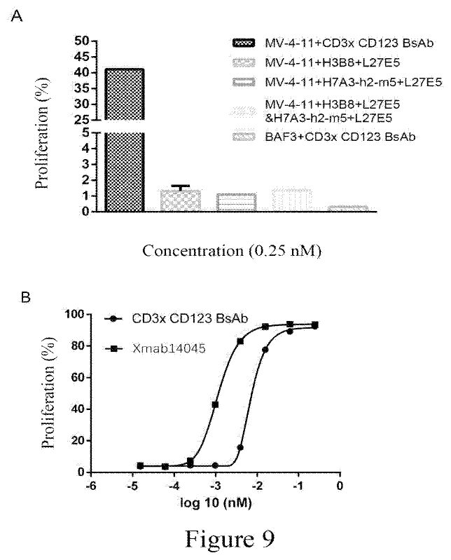

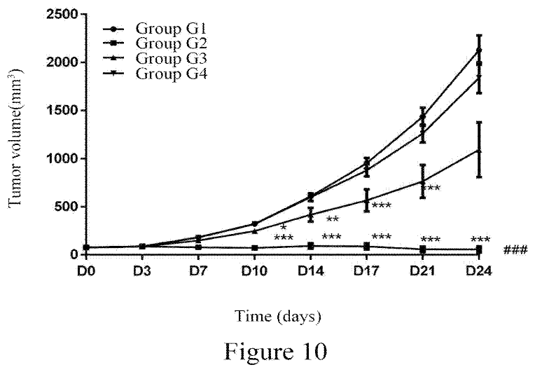

The purified T cells were obtained by sorting from PBMCs according to the instructions of the T cell negative sorting kit (BD IMaq human T lymphocyte enrichment set-DM, 557874, BD). The purified T cells were stained with reference to CFSE staining instructions (CFSE, 65-0850-84, eBioscience), resuspended in 1640 medium to 2×10⁶ cells/mL, and were added to the cell culture plate at 50 μL/well. MV-4-11 cells (CD123+ cells) at logarithmic phase of growth were collected. After centrifugation, the cells were resuspended with 1640 medium to 5×10⁵ cells/mL and plated in the cell plate at 50 μL/well. The final effector to target ratio was 4:1. Then, total 8 concentrations of the bispecific antibody CD3×CD123 (CD3×CD123 BsAb, 50 μL/well) and Xmab14045 were added with 0.25 nM as the starting concentration, followed by a 4-fold gradient dilution. Additionally, the anti-CD3E monoclonal antibody H3B8+L27E5 alone, the anti-CD123 monoclonal antibody H7A3-h2-m5+L27E5 alone, and the combination of H3B8+L27E5 and H7A3-h2-m5+L27E5 (H3B8+L27E5&H7A3-h2-m5+L27E5) were set as controls at the concentration of 0.25 nM. The bispecific antibody CD3×CD123 at a concentration of 0.25 nM in the presence of CD123-negative tumor cells (BAF3 cells) was also set as a control. After 5 days of incubation, the cells were washed twice with PBS buffer, and incubated with the anti-human CD3-APC antibody (ebioscience, 17-0037-42) at 4° C. for 30 minutes in the dark. Then, the cells were washed twice with PBS buffer, resuspended in 100 μL PBS buffer, and loaded to the flow cytometer (ACEA, Novocyte) to detect the proliferation of T cells (FIGS. 9A-B).

### Example 14: Activity Identification of the Bispecific Antibody in a CD34⁺ Mouse Model

Forty-two (32 for grouping, and 10 for spare) female hCD34+ humanized mice aged 20-24 weeks (purchased from Pharmalegacy (Shanghai)) were selected. 100 μL of 1×10⁷ MV-4-11 cells and 100 μL of Matrigel were mixed well. Then the mixture was inoculated into the right side of the back of the mice via subcutaneous injection. The mice were anesthetized with 3-4% isoflurane before inoculation. When the average tumor volume reached to about 50-80 mm³, 32 tumor-bearing mice were randomly divided into 4 groups according to the ratio of hCD45⁺ in the peripheral blood and the tumor volume, with 8 mice in each group. The day of grouping and administration was defined as day 0. The test groups were divided into 4 groups: 3 groups of the bispecific antibody CD3E×CD123 at the concentrations of 0.01 mg/kg, 0.1 mg/kg and 0.5 mg/kg, respectively; and the negative control group (IgG1m3, 0.5 mg/kg). Each group consisted of 8 mice. The antibodies were administered via tail vein injection with a total of 5 times at days 0, 3, 7, 14 and 21. The therapeutic effects were evaluated according to the relative tumor growth inhibition value (TGI), and the safety was evaluated according to the body weight change and death of mice.

During the experiment, the animals were generally in good mental states. At the end of the in vivo experiment (day 24), there was no significant difference in body weight (P>0.05) in the administration group compared with the negative control group (IgG1m3, i.v., 0.5 mg/kg, group G1). Compared with the negative control group (group G1) in the same period, the bispecific antibody CD3×CD123 at a dose of 0.5 mg/kg (group G2) significantly inhibited tumor growth, with a relative tumor inhibition rate TGI (%) of 97.35%, and 6 animals had almost complete tumor regression. There was a very significant difference between group G2 and group G1 (p<0.001). Compared with the negative control group (group G1) in the same period, after administration of the bispecific antibody CD3×CD123 at a dose of 0.1 mg/kg to the animals (group G3), the tumor volume increased slowly, and the relative tumor inhibition rate TGI (%) was 52.08%. There was a very significant difference between group G3 and group G1 (p<0.05). G4 was the group of the bispecific antibody CD3×CD123 at a dose of 0.01 mg/kg. The bispecific antibody CD3×CD123 had an obvious dose-effect relationship with tumor growth. The tumor growth of each group of animals at each time point was shown in FIG. 10. Among them, when compared with group G1, when using one-way ANOVA/Dunnett t test statistics, * represents P<0.05, ** represents P<0.01, *** represents P<0.001; when using repeated measures/Bonferroni correction statistics, ### represents P<0.001.

Although the present application has been described in detail with reference to the general description and specific embodiments, it will be apparent to those skilled in the art that modifications or improvements can be made to the present invention on the basis of the present application. Accordingly, all these modifications or improvements made without departing from the spirit of the present application will fall within the scope of the invention as claimed.

What is claimed:

1. An antibody that binds to human CD123, or an antigen-binding fragment thereof, comprising:
HCDR1 as set forth in SEQ ID NO:7,
HCDR2 as set forth in SEQ ID NO:8,
HCDR3 as set forth in SEQ ID NO:9,
LCDR1 as set forth in SEQ ID NO:4,
LCDR2 as set forth in SEQ ID NO:5, and
LCDR3 as set forth in SEQ ID NO:6;
wherein HCDRs and LCDRs are defined according to Kabat.

2. The antibody of claim 1, wherein the antibody comprises a heavy chain variable region comprising an amino acid sequence that is at least 90% identical to SEQ ID NO:13.

3. The antibody of claim 1, wherein the antibody comprises a light chain variable region comprising an amino acid sequence that is at least 90% identical to SEQ ID NO: 12.

4. The antibody of claim 1, wherein the antibody comprises a heavy chain variable region set forth in SEQ ID NO:13, and a light chain variable region set forth in SEQ ID NO: 12.

5. The antibody of claim 1, wherein the antigen-binding fragment is selected from the group consisting of a single chain fragment variable (scFv) and a Fab fragment.

6. A nucleic acid encoding the antibody or antigen-binding fragment of claim 1.

7. A host cell comprising the nucleic acid of claim 6.

8. A pharmaceutical composition comprising the antibody or antigen-binding fragment of claim 1.

9. A method of treating a CD123-positive tumor comprising administering to a subject in need thereof an efficient amount of the antibody or antigen-binding fragment of claim 1.

10. The method of claim 9, wherein the CD123-positive tumor is selected from the group consisting of acute myeloid leukemia (AML) and blastic plasmacytoid dendritic cell tumors (BPDCN).

11. An antibody that binds to human CD123, or an antigen-binding fragment thereof, wherein the antibody comprises HCDRs comprised in the heavy chain variable region set forth in SEQ ID NO: 37, and LCDRs comprised in the light chain variable region set forth in SEQ ID NO: 35.

12. The antibody of claim 11, wherein the antibody comprises a heavy chain variable region comprising an amino acid sequence that is at least 90% identical to SEQ ID NO:37.

13. The antibody of claim 11, wherein the antibody comprises a light chain variable region comprising an amino acid sequence that is at least 90% identical to SEQ ID NO:35.

14. The antibody of claim 11, wherein the antibody comprises a heavy chain variable region set forth in SEQ ID NO:37, and a light chain variable region set forth in SEQ ID NO:35.

15. The antibody of claim 11, wherein the antigen-binding fragment is selected from the group consisting of a single chain fragment variable (scFv) and a Fab fragment.

16. A nucleic acid encoding the antibody or antigen-binding fragment of claim 11.

17. A host cell comprising the nucleic acid of claim 16.

18. A pharmaceutical composition comprising the antibody or antigen-binding fragment of claim 11.

19. A method of treating a CD123-positive tumor comprising administering to a subject in need thereof an efficient amount of the antibody or antigen-binding fragment of claim 11.

20. The method of claim 19, wherein the CD123-positive tumor is selected from the group consisting of acute myeloid leukemia (AML) and blastic plasmacytoid dendritic cell tumors (BPDCN).
